(19) United States
(12) Patent Application Publication
(10) Pub. No.: US 2011/0064738 A1
(43) Pub. Date: Mar. 17, 2011

(54) HUMAN ANTI-VEGF POLYCLONAL ANTIBODIES AND USES THEREOF

(76) Inventors: Miri BLANK, Tel-Aviv (IL); Yehuda SHOENFELD, Ramat-Gan (IL)

(21) Appl. No.: 12/822,994

(22) Filed: Jun. 24, 2010

Related U.S. Application Data
(63) Continuation of application No. 12/617,603, filed on Nov. 12, 2009.
Application No. 12/617,603 is a continuation of application No. 12/149,766, filed on May 7, 2008, now abandoned.
(60) Provisional application No. 60/924,259, filed on May 7, 2007.

(51) Int. Cl. A61K 39/395; A61P 35/00

(52) U.S. Cl. 424/135.1; 424/158.1; 424/133.1

(57) ABSTRACT

This invention relates to IVIG and fragments thereof and their use, specifically, provided herein are compositions and methods of inhibiting VEGF or VEGF receptor using polyclonal antibodies (pAb), or fragments thereof derived from human immunoglobulins.

### CROSS-REFERENCE TO RELATED APPLICATION

This application is a continuation of U.S. patent application Ser. No. 12/617,603 filed on Nov. 12, 2009, which is a continuation of U.S. patent application Ser. No. 12/149,766 filed May 7, 2008, abandoned, which claims priority under 35 U.S.C. 119(e) to U.S. Provisional Patent Application Ser. No. 60/924,259, filed May 7, 2007, all of which are incorporated herein by reference in their entireties.

### FIELD OF INVENTION

This invention is directed to antibodies specific against vascular endothelial growth factor (VEGF) or its receptors, and their uses, specifically, provided herein are compositions and methods of inhibiting VEGF or its receptors using polyclonal antibodies (pAb), or fragments thereof derived from human immunoglobulins.

### BACKGROUND OF THE INVENTION

Intravenous immunoglobulin (IVIG) is a safe preparation made from sterilized purified human plasma harvested from thousands of healthy donors. Therefore, IVIG most likely contains the entire wide spectrum of variable regions present in normal plasma. IVIG contains more than 95% intact immunoglobulin G (IgG) molecules, with traces of immunoglobulin A (IgA) (less than 2.5%) and immunoglobulin M (IgM) (negligible). The IgG subclasses distribution corresponds to that of normal human plasma. During the last two decades, IVIG has shown potent immunomodulatory and anti-inflammatory effects in immune deficiencies, infections, a wide range of autoimmune diseases (e.g. idiopathic thrombocytopenic purpura, systemic lupus erythematosus, neurological disorders such as Guillain-Barre syndrome (GBS), connective tissue disorders), as well as several cancers (e.g. colon cancer, melanoma and others).

Angiogenesis refers to the formation of new blood vessels during embryonic development, in normal physiology and in pathological conditions. Angiogenesis is critical for the development and subsequent growth of human tumors and is a prerequisite for the formation of metastases. Various pro-angiogenic factors secreted by tumor cells and/or host factors stimulate endothelial cells to proliferate and to form new, qualitatively poor and often leaky new blood vessels. As few as 60-200 tumor cells can initiate the process of angiogenesis. Although various pro-angiogenic factors such as basic fibroblast growth factor (bFGF) and platelet derived growth factor (PDGF) are involved, the vascular endothelial growth factor (VEGF) family, and especially isoform VEGF-165, is the predominant proangiogenic factor. VEGF is distinguished from other angiogenic factors by being the only one whose synthesis is regulated by oxygen availability, therefore it is an important stimulator of abnormal to angiogenesis in cancer.

In addition to cancer, VEGF family members play a role in ocular neovascular disorders, including age-related macular degeneration, pathologic myopia and diabetic retinopathy.

VEGF was identified as a protein that induces proliferation and migration of endothelial cells in vitro, and blood vessel permeabilization and angiogenesis in vivo. It regulates both vascular proliferation and permeability. Also known as vascular permeability factor (VPF), it is unique among pro-angiogenic factors because of its specificity for vascular endothelium and potency. It also functions as an anti-apoptotic factor for endothelial cells in newly formed vessels. VEGF is expressed in tumor cells, macrophages, T cells, smooth muscle cells, kidney cells, mesangial cells, keratinocytes, astrocytes, and osteoblasts.

The VEGF family comprises seven members, including VEGF-A, VEGF-B, VEGF-C, VEGF-D, VEGF-E, VEGF-F, and placenta growth factor (PIGF). All of them have a common structure of eight cysteine residues in a VEGF homology domain. In addition, in relation to VEGF-A, there are six different isoforms, and VEGF-A165 is the main isoform. All these isoforms have distinct and overlapping functions in angiogenesis. The VEGF gene is located on chromosome 6p. 21. The different members of VEGF family have different physical and biological properties and they act through specific tyrosine kinase receptors (VEGFR-1, VEGFR-2, and VEGFR-3). The VEGFR-3 receptor and its ligands, VEGF-C and VEGF-D, are associated with lymphangiogenesis, while PIGF is linked to arteriogenesis.

VEGF inhibition can be an attractive therapeutic strategy because it is highly specific and may be less toxic than cytotoxic therapy. VEGF inhibitors offer a means to control a heterogeneous tumor population by influencing a relatively homogeneous endothelial population. VEGF inhibitors should control tumor growth independent of specific tumor details and induce a dormant state in which pro-angiogenic and anti-angiogenic factors come back into balance and tumor growth is controlled. Therefore, anti-VEGF antibodies or other inhibitors of VEGF action are promising candidates for the treatment of solid tumors and various other neovascular disorders. Likewise, inhibiting VEGF receptor (VEGFR) activity can potentially achieve the same biological effects as direct inhibition of VEGF.

### SUMMARY OF THE INVENTION

In one embodiment, the invention provides a method for inhibiting angiogenesis in a subject, comprising the step of administering to the subject a preparation of polyclonal antibodies (pAb), or fragments thereof in an amount sufficient to inhibit metastasis, whereby said preparation binds or inhibits VEGF.

In another embodiment, provided herein is a method for inhibiting vascular proliferation and permeability in a subject, comprising the step of administering to the subject a preparation of polyclonal antibodies (pAb) or fragments thereof in an amount sufficient to inhibit metastases whereby said preparation binds or inhibits VEGF.

In one embodiment, provided herein is a composition for inhibiting neovascularization comprising a preparation of polyclonal antibodies (pAb), or fragments thereof, and a pharmaceutically acceptable carrier, excipient, flow agent, processing aid, diluent or a combination thereof, wherein said preparation or fragments thereof is specific against VEGF.

In another embodiment, the invention provides a method for inhibiting angiogenesis in a subject, comprising the step of administering to the subject a preparation of polyclonal antibodies (pAb), or fragments thereof in an amount sufficient to inhibit metastasis, whereby said preparation binds and inhibits VEGF receptors.

In one embodiment, provided herein is a method for inhibiting vascular proliferation, permeability or their combination in a subject, comprising the step of administering to the subject a preparation of polyclonal antibodies (pAb), or fragments thereof in an amount sufficient to inhibit metastases whereby said preparation inhibits VEGF receptors.

In another embodiment, provided herein is a composition for inhibiting neovascularization comprising a preparation of polyclonal antibodies (pAb), or fragments thereof, and a pharmaceutically acceptable carrier, excipient, flow agent, processing aid, diluent or a combination thereof, wherein said preparation or fragments thereof is specific against one or a combination of VEGF receptors.

### BRIEF DESCRIPTION OF THE DRAWINGS

FIG. 1 shows a schematic representation of a “sandwich” ELISA. Capture of specific antigen (recombinant human VEGF) was achieved using mouse anti-human VEGF antibody coated on to ELISA plate, followed by biotinylated IVIG, utilized as detection antibody. Visualization was enabled by streptavidin conjugated to HRP, followed by addition of is appropriate substrate;

FIG. 2 shows binding of IVIG to recombinant VEGF in a “sandwich” ELISA (A) and in an immunoblot (B). (A) For ELISA measurements, pre-coated mouse anti-human VEGF plates were used to capture a complex consisting of recombinant human VEGF and biotinylated IVIG. (B) For immunobloting, VEGF samples were loaded onto a 12% SDS-PAGE gel and blotted onto nitrocellulose membranes. The membrane was incubated for four hours with two concentrations of biotinylated IVIG, 2 mg/ml (left line) and 2 μg/ml (right line) followed by the application of SA-HRP conjugated secondary antibody;

FIG. 3 shows anti-VEGF activity in a direct solid-phase immobilized VEGF ELISA testing. VEGF was coated on an ELISA plate at 0.5 μg/ml. Serial dilutions of biotinylated IVIg were prepared and binding to VEGF was detected using streptavidin conjugated to HRP followed by a substrate;

FIG. 4 shows the blocking of anti-VEGF activity in direct ELISA- and immunoblot-based binding assays;

FIG. 5 shows the competition between a commercial human monoclonal antibody against VEGF and IVIg for binding to VEGF in an immunoblot; and

FIG. 6 shows the anti-VEGF activity of IVIg in a mouse hind-limb ischemia model.

### DETAILED DESCRIPTION OF THE INVENTION

This invention relates in one embodiment to anti-VEGF antibodies and their use, specifically, provided herein are compositions and methods of inhibiting VEGF using polyclonal antibodies (pAb), or fragments thereof derived from human immunoglobulins.

In one embodiment, VEGF refers to a gene family that comprises seven members, including VEGF-A, VEGF-B, VEGF-C, VEGF-D, VEGF-E, VEGF-F, and placenta growth factor (PIGF) that share a common structure of eight cysteine residues in a VEGF homology domain and all of which are regulators of angiogenesis or lymphangiogenesis or of both processes.

In another embodiment VEGF is the most important inducer of angiogenesis. This is primarily because VEGF is distinguished from other angiogenic factors by being the only one whose synthesis is regulated by oxygen availability, therefore an important stimulator of abnormal angiogenesis in cancer. Angiogenesis is the basic process leading to tumor spread and metastasis.

The role of VEGF in angiogenic processes is evident in one embodiment from gene targeting showing that even the absence of one allele of VEGF causes severe disruptions in the development of the vasculature, leading to early lethality. In one embodiment, VEGF increases the permeability of blood vessels to large proteins such as globulins and fibrinogen. This property is of central importance to tumor development since the fibrin matrix that forms in tumors following leakage from tumor associated vessels provides a matrix that supports the proliferation of the tumorigenic cells.

In certain embodiments, hypoxia regulates VEGF production through the HIF-1α transcription factor. The levels of HIF-1α in turn are regulated by prolyl-hydroxylases, the activity of which is directly regulated by the availability of oxygen. The central regions of solid ors become hypoxic when tumors expand, thus promoting VEGF production and induction of tumor angiogenesis in another embodiment. VEGF expression and VEGF induced tumor angiogenesis are induced, in one embodiment, following the activation of oncogenes such as ras in tumorigenic cells. In one embodiment, the compositions and methods provided herein are useful in the treatment of pathologies associated with activation of VEGF.

According to this aspect of the invention and in one embodiment, provided herein is a method for inhibiting angiogenesis in a subject, comprising the step of administering to the subject a preparation of polyclonal antibodies (pAb), or fragments thereof in an amount sufficient to inhibit metastasis, whereby said preparation binds or inhibits VEGF.

The term “human VEGF” refers in one embodiment to the 165-amino acid human vascular endothelial cell growth factor, and related 121-, 189-, and 206-amino acid vascular endothelial cell growth factors, as described by Leung et al., Science 246:1306 (1989), and Houck et al., Mol. Endocrin. 5:1806 (1991) together with the naturally occurring allelic and processed forms of those growth factors.

Provided herein are compositions and preparations comprising anti-VEGF antagonistic antibodies, capable of inhibiting one or more of the biological activities of VEGF, such as its mitogenic activity in one embodiment, or angiogenic activity in another embodiment. VEGF antagonists act in one embodiment, by interfering with the binding of VEGF to a cellular receptor, or by interfering with vascular endothelial cell activation after VEGF binding to a cellular receptor in other embodiments.

In another embodiment, provided herein is a method for inhibiting vascular proliferation and permeability in a subject, comprising the step of administering to the subject a preparation of polyclonal antibodies (pAb) or fragments thereof in an amount sufficient to inhibit metastases whereby said preparation binds or inhibits VEGF.

Two high affinity receptors for VEGF have been characterized, VEGFR-1/Flt1 (fms-like tyrosine kinase-1) and VEGFR-2/Kdr/Flk-1 (kinase insert domain containing receptor/fetal liver kinase-1). A third receptor, VEGFR-3 is also known. These receptors are classified in the PDGF-receptor family. However, the VEGF receptors have seven immunoglobulin-like loops in their extracellular domains (as opposed to five in other members of the PDGF family) and a longer kinase insert. The expression of VEGF receptors occurs mainly in vascular endothelial cells, although some may also be present on monocytes and on melanoma cell lines. Only endothelial cells have been reported to proliferate in response to VEGF, and endothelial cells from different sources show different responses. Thus, the signals mediated through VEGFR-1, VEGFR-2 and VEGFR-3 appear to be cell type specific.

VEGFR-1 and VEGFR-2 bind VEGF 165 with high affinity (K_{d} about 20 pM and 200 pM, respectively). Flk-1 receptor has also been shown to undergo autophosphorylation in response to VEGF, but phosphorylation of Flt1 was barely detectable. VEGFR-2 mediated signals cause striking changes in the morphology, actin reorganization and membrane ruffling of porcine aortic endothelial cells overexpressing this receptor. In these cells, VEGFR-2 also mediated ligand-induced chemotaxis and mitogenicity; whereas VEGFR-1 transfected cells lacked mitogenic responses to VEGF. In contrast, VEGF had a strong growth stimulatory effect on rat sinusoidal endothelial cells expressing VEGFR-1. Phosphoproteins co-precipitating with VEGFR-1 and VEGFR-2 are distinct, suggesting that different signalling molecules interact with receptor specific intracellular sequences.

Abundant VEGFR-2 mRNA in proliferating endothelial cells of vascular sprouts and branching vessels of embryonic and early postnatal brain and decreased expression in adult brain indicates that in one embodiment VEGFR-2 is a major regulator of vasculogenesis and angiogenesis. VEGFR-1 expression is associated in another embodiment with early vascular development in mouse embryos and with neovascularization in healing skin wounds. In one embodiment, high levels of VEGFR-1 expression detected in adult organs, indicate that VEGFR-1 has a function in quiescent endothelium of mature vessels not related to cell growth.

In one embodiment, antibodies that “specifically inhibit” VEGFR-2 (KDR/Flk-1) or VEGFR-1 receptor (Flt-1) or VEGFR-3, or a combination thereof refers to inhibition by competition in one embodiment, and/or functionality, in another embodiment. In another embodiment, “inhibits” refers to a compound acting as an antagonist of the VEGFRs. The term “antagonist” refers in another embodiment to both full antagonists and partial antagonists, as well as inverse agonists in other embodiments of the pAbs provided herein. In another embodiment, “VEGF receptor antagonists” refers to compounds which selectively antagonize, inhibit or counteract binding of a physiological ligand to the VEGFR-1, to compounds which selectively antagonize, inhibit or counter-act binding of a physiological ligand to the VEGFR-2, inhibit or counter-act binding of a physiological ligand to the VEGFR-3, to compounds which antagonize, inhibit or counteract binding of a physiological ligand to all three or a combination of the VEGFR-1, VEGFR-2 and VEGFR-3.

The term “Antibodies” (Abs) and “immunoglobulins” (IgGs) refers in one embodiment to glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas.

In one embodiment, the term “antibody” includes complete antibodies (e.g., bivalent IgG, pentavalent IgM) or fragments of antibodies in other embodiments, which contain an antigen binding site. Such fragments include in one embodiment Fab, F(ab′)₂, Fv and single chain Fv (scFv) fragments. In one embodiment, such fragments may or may not include antibody constant domains. In another embodiment, F(ab)'s lack constant domains, which are required for complement fixation. scFvs are composed of an antibody variable light chain (V_{L}) linked to a variable heavy chain (V_{H}) by a flexible linker. ScFvs are able to bind antigen and can be rapidly produced in bacteria. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

The antibodies described herein can be monoclonal antibodies (Mab) in one embodiment, or polyclonal antibodies in another embodiment. Antibodies of the invention that are useful for the compositions, methods and described herein can be from any source, and in addition may be chimeric. In one embodiment, sources of antibodies can be from a mouse, or a rat, or a human in other embodiments. Antibodies of the invention that are useful for the compositions, methods and of the invention have reduced antigenicity in humans, and in another embodiment, are not antigenic in humans.

In one embodiment, the antibody, a fragment thereof, or their combination, exhibit substantial complimentarity to their target sequence, which may be a protein, such as a VEGF protein or a VEGFR protein. In another embodiment, “complementary” indicates that the oligopeptide described in the compositions herein and is used in the methods provided herein, has a base sequence containing at least 15 contiguous base region that is at least 70% complementary, or in another embodiment at least 80% complementary, or in another embodiment at least 90% complementary, or in another embodiment 100% complementary to an-at least 15 contiguous amino acid region present on a target protein sequence.

As will be understood by those skilled in the art, the immunologically binding reagents encompassed by the term “antibody” extend in certain embodiments, to all antibodies from all species including dimeric, trimeric and multimeric antibodies; bispecific antibodies; chimeric antibodies; human and humanized antibodies; recombinant and engineered antibodies, and fragments thereof. The term “antibody” is refers in another embodiment to any antibody-like molecule that has an antigen binding region, and this term includes antibody fragments such as Fab′, Fab, F(ab′).sub.2, single domain antibodies (DABs), Fv, scFv (single chain Fv), linear antibodies, diabodies, and the like. The techniques for preparing and using various antibody-based constructs and fragments are well known in the art (see Kabat et al., 1991, specifically incorporated herein by reference). In one embodiment, the anti-VEGF fragment used in the methods and compositions described herein, is Fc, or Fab, F(ab′), F(ab′)₂ or a combination thereof in other embodiments. In another embodiment, the anti-VEGFR fragment used in the methods and compositions described herein, is Fc, or Fab, F(ab′), F(ab′)₂ or a combination thereof in other embodiments.

The term “antibody fragment” also includes any synthetic or genetically engineered protein that acts like an antibody by binding to a specific antigen to form a complex. In one embodiment, antibody fragments include isolated fragments, “Fv” fragments, consisting of the variable regions of the heavy and light chains, recombinant single chain polypeptide molecules in which light and heavy chain variable regions are connected by a peptide linker (“sFv proteins”), and minimal recognition units consisting of the amino acid residues that mimic the hypervariable region. In one embodiment, the antibody capable of inhibiting human VEGF or human VEGFR is a variable regions of the heavy and light chains, or recombinant single chain polypeptide molecules in which light and heavy chain variable regions are connected by a peptide linker (“sFv proteins”), and minimal recognition units consisting of the amino acid residues that mimic the hypervariable region in other embodiments.

In one embodiment, the preparation of IVIG or fragments thereof, used in the compositions and methods described herein is administered intravenously, intracavitarily, subcutaneously, intratumorally, or a combination thereof.

“Intracavitary administration”, as used herein, refers to administering a substance directly into a body cavity of a mammal. Such body cavities include the peritoneal cavity, the pleural cavity and cavities within the central nervous system, including the orbit of the eye.

The dosage of IVIG and the method of administration will vary with the severity and nature of the particular condition being treated, the duration of treatment, the adjunct therapy used, the age and physical condition of the subject of treatment and like factors within the specific knowledge and expertise of the treating physician. However, single dosages for intravenous and intracavitary administration can typically range from 4 mg to 2 g per kilogram body weight, preferably 2 g/kg (unless otherwise indicated, the unit designated “mg/kg” or “g/kg”, as used herein, refers to milligrams or grams per kilogram of body weight). The preferred dosage regimen is 400 mg/kg/day for five consecutive days per month or 2 g/kg/day once a month. The IVIG preparation described herein are effective in another embodiment in inhibiting metastasis when administered by intravenous or intraperitoneal injection and in the dose range of 1-1000 mg/kg/week.

In another embodiment of this invention, the IVIG preparation is administered via the subcutaneous route. The typical dosage for subcutaneous administration can range from 4 mg to 20 mg per kg body weight. According to the present invention IVIG may be administered as a pharmaceutical composition containing a pharmaceutically acceptable carrier. The carrier must be physiologically tolerable and must be compatible with the active ingredient. Suitable carriers include, sterile water, saline, dextrose, glycerol and the like. In addition, the compositions may contain minor amounts of stabilizing or pH buffering agents and the like. The compositions are conventionally administered through parenteral routes, with intravenous, intracavitary or subcutaneous injection being preferred.

The preparation comprising the polyclonal antibodies described herein, or their fragments; are administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual subject, the site of delivery, the method of administration and other factors known to practitioners. Examples of techniques and protocols can be found in Remington's Pharmaceutical Sciences.

In one embodiment, the methods provided herein, using the compositions described herein, further comprise subjecting the subject to at least one other treatment modality, prior to, during or after the administration of the preparation of polyclonal antibodies (pAb) or fragments thereof. That treatment modality is, in another embodiment, chemotherapy, immunotherapy, radiation therapy, surgery or a combination thereof, in certain other embodiments.

In one embodiment, the methods described herein use the compositions described herein. According to this aspect of the invention and in one embodiment, provided herein is a composition for inhibiting neovascularization comprising a preparation of polyclonal antibodies (pAb), or fragments thereof, and a pharmaceutically acceptable carrier, excipient, flow agent, processing aid, diluent or a combination thereof, wherein said preparation or fragments thereof is specific against VEGF or VEGFR. In one embodiment, the compositions described herein are in a form suitable for oral administration, or intravenous, intratumoral, intraarterial, intramuscular, subcutaneous, parenteral, transmucosal, transdermal, ocular or topical administration, or a combination thereof in other embodiments.

In one embodiment, the composition for inhibiting neovascularization comprising a preparation of polyclonal antibodies (pAb), or fragments thereof is a topical application in the form of a cream, an ointment, a suspension, an emulsion, a gel or a combination thereof.

In one embodiment, the carrier, excipient, lubricant, flow aid, processing aid or diluent used in the compositions described herein is a gum, a starch, a sugar, a cellulosic material, an acrylate, calcium carbonate, magnesium oxide, talc, lactose monohydrate, magnesium stearate, colloidal silicone dioxide or mixtures thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitoneally, intraventricularly, or intracranially.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraarterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions of the invention are administered in conjunction with other therapeutic agents. In one embodiment, the therapeutic agent administered in conjunction with the compositions provided herein, is an additional anti-VEGF antibody, such as bevacizumab, or a rat-humanized monoclonal antibody specific against VEGF ranibizumab (rhuMabVEGF), or their combination in other embodiments.

In one embodiment, the compositions described in the embodiments hereinabove, are used in the methods provided herein. According to this aspect of the invention and in one embodiment, provided herein is a method of inhibiting a tumor growth in a subject, or treating neovascular disorder in another embodiment, comprising the step of administering to the subject the composition described herein.

The intravenous immunoglobulins administered according to the present invention act as antiangiogenic, antineoplastic or antimetastatic agents resulting in the reduction of tumor cell migration, tumor colony number as well as tumor colony size. They can also act prophylactically i.e., to prevent metastasis of tumors. The intravenous immunoglobulins according to this invention may also be used to reduce the size of the primary tumor.

In one embodiment, the preparation of polyclonal antibodies (pAb), or fragments thereof, used in the methods and compositions described herein, is isolated from the plasma of a pool of subjects, wherein said pool of subjects are healthy, in cancer remission, or a combination thereof. In one embodiment, the gamma globulins may be prepared from the whole blood of one or more donors, preferably from a plurality of donors. In certain embodiments, the donors comprise mature members of the species so as to assure that each donor has been exposed to a number of different antigens during their lifetime and will thus have developed immunity against a variety of antigens. The use of a plurality of donors increases in another embodiment, the type and number of different gamma globulins obtained from the collected blood.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combination thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

In one embodiment, the neovascular disorder treated using the methods and compositions described herein is ocular neovascular disorder, age-related macular degeneration (ARMD or AMD), pathologic myopia, pterigium or a combination thereof.

In one embodiment, the compositions and methods provided herein are useful in preventing and treating any ocular neovascularization, including, but not limited to: retinal diseases (diabetic retinopathy, chronic glaucoma, retinal detachment, sickle cell retinopathy, senile macular degeneration due to subretinal neovascularization); rubeosis iritis; inflammatory diseases; chronic uveitis; neoplasms (retinoblastoma, pseudoglioma); Fuchs' heterochromic iridocyclitis; neovascular glaucoma; corneal neovascularization (inflammatory, transplantation, developmental hypoplasia of the iris); neovascularization resulting following a combined vitrectomy and lensectomy; vascular diseases (retinal ischemia, choroidal vascular insufficiency, choroidal thrombosis, carotid artery ischemia); pterigium; neovascularization of the optic nerve; and neovascularization due to penetration of the eye or contusive ocular injury.

Neovascular diseases of the retina include diabetic retinopathy, age-related macular degeneration, neovascular glaucoma, retinopathy of prematurity, sickle-cell retinopathy, retinal vein occlusion, oxygen induced retinopathy, and neovascularization due to ocular insults such as traumatic or surgical injury, or transplantation of eye tissue. Other conditions or diseases associated with the manifestation of retinal neovascularization include any disease or condition where a part of the retina is subject to a relatively non-perused state compared to surrounding tissue, where any one or more of the proteins, proteinases, hormones, or cellular signals associated with angiogenesis are detected, or where new vessel growth can be detected or observed. In addition, diseases implicating matrix metalloproteinase activity, endothelial invasion, or the generation of new blood vessels may also be associated with retinal neovascularization according to this invention.

Neovascularization involves both the degradation of tissue through enzymatic action and the formation of new tissue. A crucial event in the retinal neovascularization process is the migration of epithelial cells, which involves proteolysis of basement membrane components, typically by one or more proteinases. At active neovascularization sites, both the high (54 kD) and low (33 kD) molecular weight forms of the protein urokinase have been found at levels significantly higher than in normal retinas. The levels of both pro and active forms of the matrix metalloproteinases (MMPs) MMP-2 (gelatinase) and MMP-9 are also significantly elevated in neovascular membranes in comparison to normal retinas. In certain embodiments, the active forms of MMPs such as collagenase, stromelysin and gelatinase are not present at detectable levels in normal retinas.

Age-related macular degeneration is one of the leading causes of blindness in older adults in the United States, and may account for up to 30 percent of all bilateral blindness among Caucasian Americans. This disease is characterized by loss of central vision, usually in both eyes, due to damage to the retinal pigment epithelial (RPE) cells. RPE cells are aligned in the lowest layer of the retina, on the Bruch's membrane, and absorb the light that reaches the retina so as to prevent reflection. RPE cells also constitute the blood-retinal barrier, which partitions the visual cells and the vascular layer of choroid together with the Bruch's membrane. In general, RPE cells have important physical and physiological functions, such as sustainment and regeneration of visual cells.

Pterygium is a condition characterized by a triangular or wing-shaped overgrowth of abnormal conjunctiva onto the cornea, and it is prevalent in per equatorial and tropical regions. In severe cases, visual loss may arise from induced irregular corneal astigmatism, corneal stromal scarring, and obscuration of the visual axis, while ocular irritation often occurs as a result of ocular surface inflammation at the site of the pterygium. Pterygium is of great concern to both surgeons and patients as it has been shown to recur in up to 97% of patients within 1 year after surgical removal. In one embodiment particular molecular alterations such as the activation of oncogenes, or the aberrant expression of growth factors such as VEGF, play an important role in the development of the disease. Therefore, in one embodiment, the compositions described herein are useful in the post operative treatment of pterygium, or in its inhibition.

In one embodiment, VEGF family members have a role in pathological conditions that are associated with autoimmune diseases such as, in another embodiment, systemic lupus erythematosus (SLE), or rheumatoid arthritis (RA), or multiple sclerosis (MS) in other embodiments. In one embodiment, VEGF serum levels correlate with disease activity in a large number of autoimmune diseases and fall with the use of standard therapy. Accordingly and in one embodiment, the IVIG described hereinabove is effective in treating, inhibiting or suppressing, or reducing symptoms associated with autoimmune diseases, such as SLE, MS, RA and the like.

Likewise and in another embodiment, VEGFR family members have a role in pathological conditions that are associated with autoimmune diseases. In one embodiment, VEGF serum levels correlate with disease activity are regulated through the various family members of VEGFR in a large number of autoimmune diseases. In another embodiment, the expression of ADAM15 in RA is up-regulated by the action of VEGF₁₆₅ via VEGFR-2, or in another embodiment, patients with active SLE show levels of VEGF and sVEGFR-1 that are higher than in patients with inactive SLE.

In one embodiment, the mechanisms action of VEGF family members involves overlapping pathways and cross-talk between other receptors such as the neuropilins Neuropilins are multifunctional non-tyrosine kinase receptors that bind to class 3 semaphorins and vascular endothelial growth factor. NRP-1 and NRP-2 were first identified for their role in mediating axonal guidance in the developing nervous system through their interactions with class 3 semaphorins. In one embodiment neuropilins receptors have a critical role in tumor progression. In another embodiment, neuropilin expression is up-regulated in multiple tumor types, and correlates with tumor progression and prognosis in specific tumors. In one embodiment, neuropilins indirectly mediate effects on tumor progression by affecting angiogenesis, or in another embodiment, directly through effects on tumor cells.

In one embodiment, neuropilin is a co-receptor for some of the isoforms of the vascular endothelial growth factor (VEGF) family. The presence of Neuropilin on endothelial or other cells increases in another embodiment, the binding of these isoforms to their signaling receptor VEGFR2, thus increasing pro-angiogenesis signaling and stimulating vascular growth. In one embodiment, the IVIG described hereinabove effects its inhibitory activity on VEGF by binding to the neuropilins on endothelial and other cell types.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

### Immunoglobulins

IVIG—IVIG preparation used in the study was kindly provided by Omrix Biopharmaceuticals Inc., Ness-Ziona, Israel.

Normal human IgG—IgG affinity purified from serum of healthy single subject on a protein G column (Pharmacia Fine Chemicals, Uppsala, Sweden) according to the manufacturer's instructions.

### Recombinant VEGF

Recombinant VEGF was obtained from ProSpec-Tany TechnoGene, Rehovot, Israel. Recombinant Human VEGF produced in Ecolab is a double, non-glycosylated, polypeptide chain containing 165 amino acids and having a molecular mass of 38231 Dalton.

### Biotinylation of Antibody

Biotinylation was done in 100 mM carbonate buffer, pH 8.5 using biotin-amidohexanoic acid NHS (Sigma, St. Louis, USA). The molecular ratio biotin:protein was 10:1.

### Anti-VEGF Sandwich ELISA

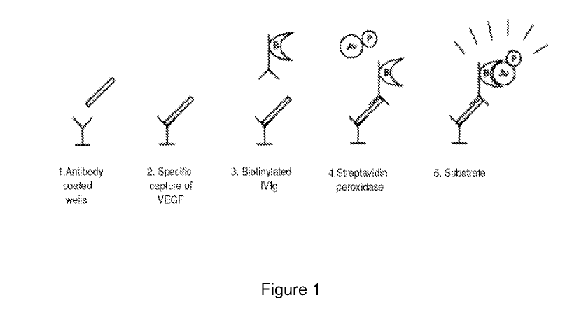

The presence and the level of anti-VEGF activity in an IVIG preparation was determined using a quantitative sandwich enzyme immunoassay technique (DuoSet ELISA Development R & D Systems), based on the use of biotinylated IVIG sample instead of biotinylated anti-VEGF detection antibody, included in the kit as the component part (as stated in FIG. 1).

Briefly, plates were coated with 1 μg/ml mouse anti-VEGF antibody, then washed and blocked before adding recombinant human VEGF. Biotinylated IVIG was used to detect bound VEGF and streptavidin-peroxidase was added to amplify the antibody±antigen reaction. Color was developed using tetramethylbenzidine (TMB) and absorbance was read at a wavelength of 450 nm on a microtiter plate reader.

### Direct ELISA

Recombinant human VEGF was used at concentration of 0.5 μg/ml in phosphate-buffered saline for coating ELISA plates at 4° C. overnight. After washing with PBS containing 0.05% Tween 20, plates were blocked with 3% BSA. Serial dilutions of either biotinylated IVIG or anti-VEGF monoclonal antibody or single person IgG were added to the plates for 2 hours. The plates were washed three times. Streptavidin-peroxidase (from the anti-VEGF sandwich ELISA kit) was incubated in plates for 20 minutes. After washing, TMB and appropriate substrate was added in the plate for color development. Absorbance (450 nm) was read for quantification of antibody-binding activity.

### Anti-VEGF Binding by Immunoblot

Recombinant VEGF samples were loaded onto 12% sodium dodecyl sulphate polyacrylamide gel electrophoresis (SDS-PAGE) (1 μg/lane) and separated under non-reducing conditions. The protein was transferred to nitrocellulose. The membrane was blocked with 10% skim-milk in TBS (20 mM Tris-HCl, 150 mM NaCl, pH 8.0) overnight at 4° C. Biotinylated anti-VEGF monoclonal antibody or IVIG in TBS 0.1% Tween-20 and 0.5% skim milk, at different concentrations, were added for 2 hours incubation at room temperature. In several inhibition experiments, the VEGF nitrocellulose strips were pre-incubated either with non-biotinylated anti-VEGF monoclonal antibody or IVIG for 2 hours at room temperature (e.g. inhibition of biotinylated IVIG binding to VEGF by anti-VEGF monoclonal antibody or inhibition of biotinylated Anti-VEGF monoclonal antibody binding to VEGF by non-biotinylated IVIG). The binding was probed streptavidin-peroxidase followed by ECL-Luminol Reagent according to the instructions of the manufacturer (Santa Cruz Biotechnology, Inc, Santa Cruz, Ca).

### Example 1

### Determination of Anti-VEGF Activity in IVIG by a Sandwich ELISA

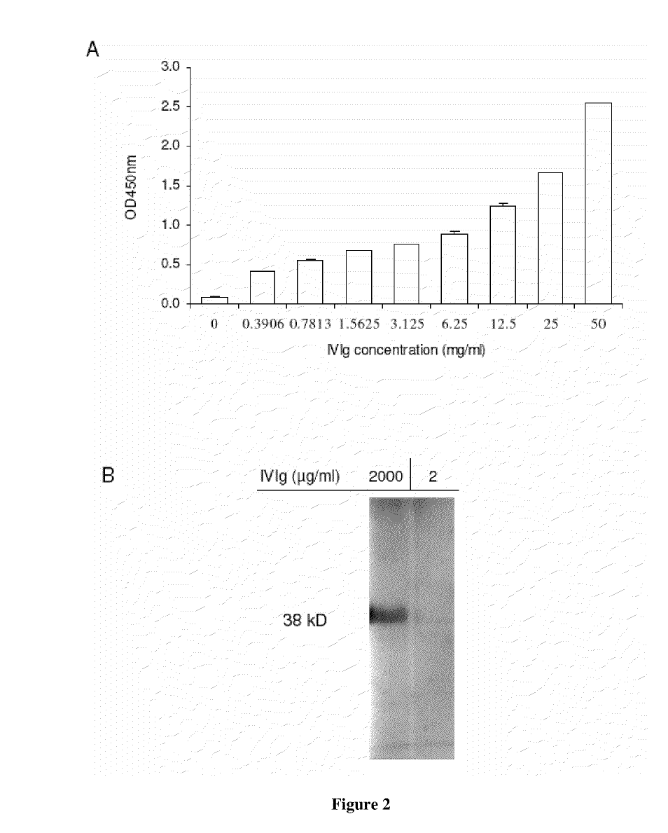

Anti-VEGF antibody levels in an IVIG preparation were determined using a quantitative sandwich ELISA as described in FIG. 1. Results are shown in FIG. 2. A typical dose-response curve was obtained by plotting dilution rate of IVIG versus absorbance at 450 nm (FIG. 2A). The calculated 50% effective concentration (EC₅₀) value for IVIG was approximately 12.5 mg/ml. Intra-assay variations were estimated by duplicate measurements and at least three independent experiments.

Performance of a Western immunoblot assay to detect specific anti-VEGF antibodies in an IVIG preparation revealed an intense positive band migrating around 38 kDa, which corresponds to the molecular mass of VEGF. Strong, positive reaction was noticed using 2 mg/ml of IVIG whereas a lower concentration of 2 μg/ml did not yield a clear pattern of binding (FIG. 2B).

It is important to point out that in the sandwich ELISA used, in control wells without VEGF, some reactivity between IVIG and mouse anti-VEGF capture antibody was noticed also (data not shown). Therefore, to confirm the interaction between IVIG and VEGF, additional experiments were performed, using an ELISA assay with solid-phase immobilized VEGF.

### Example 2

### Direct Binding of IVIG to VEGF in a Solid-Phase Immobilized VEGF ELISA

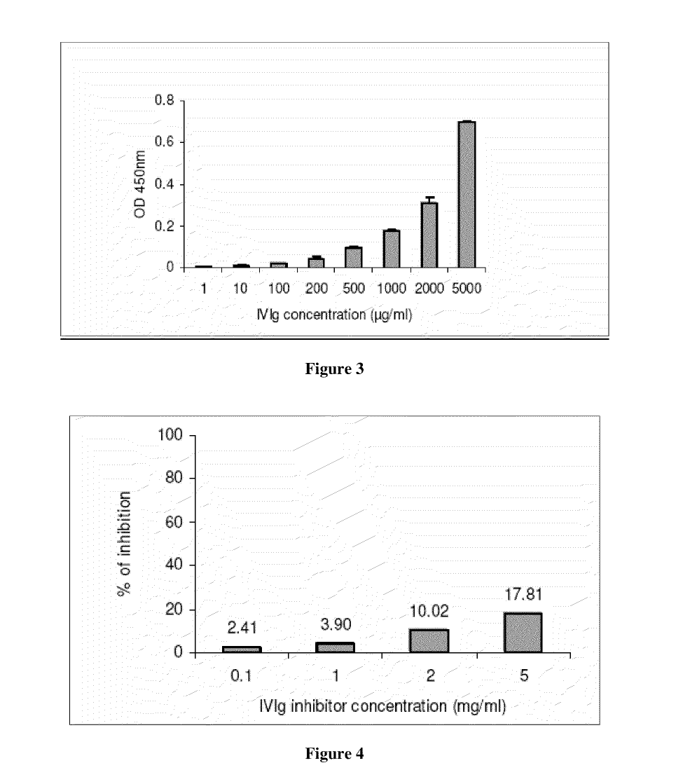

To further confirm an anti-VEGF activity in the IVIG, plates where VEGF was attached to a solid surface were used for ELISA testing (FIG. 3). The results presented in FIG. 3 revealed a dose-dependent direct binding of IVIG to VEGF, thus confirming the results obtained by sandwich ELISA (see above). The minimum detection limit estimated by serial dilutions was about 10 μg/ml and the EC₅₀ was found to be 2.35 mg/ml. For validation of specificity and sensitivity of IVIG binding, biotinylated commercial monoclonal anti-VEGF antibody was employed as positive control. Anti-VEGF antibody bound to VEGF coated plates in a dose-dependent fashion and its minimum detection limit estimated by serial dilutions was about 10 ng/ml. Determined EC₅₀ value was about 5 μg/ml and should be used as concentration of inhibitor in an IVIG-to-VEGF inhibition assay.

Next the relative binding capabilities of the IVIG were compared with those of anti-VEGF monoclonal antibody and single person IgG (used as positive and negative controls, respectively) to bind immobilized VEGF. For that purpose, all three preparations were used at the same concentration of 10 μg/ml and 1 μg/ml. As expected, anti-VEGF monoclonal antibody exhibited much greater binding potential than those of IVIG. On the other hand, for all tested concentrations levels of IVIG binding were significantly higher than were those of single person IgG, which, otherwise, showed a minimal activity.

All results obtained from direct ELISA tests indicated that coating of microtiter plate wells with 0.5 μg/ml VEGF was sufficient for maximal binding signals (FIG. 3).

### Example 3

### Inhibition of IVIG-VEGF Binding with VEGF

To assess binding specificity of IVIG-to-VEGF, ELISA inhibition studies were performed. Preincubation of biotinylated IVIG with different concentrations of unlabeled, soluble VEGF, as inhibitor, resulted in inhibition of IVIG-to-VEGF binding in the sandwich ELISA. Results in Table 1 show that all tested concentrations of the soluble VEGF were able, in a dose responsive manner, to inhibit the binding of IVIG-to-VEGF captured by an anti-VEGF antibody. The VEGF inhibitor used at concentrations of 8-, 4-, and 2 ng/ml give rise to 95.2, 91.6, and 88.7% of inhibition, respectively.

TABLE 1
Inhibition of IVIg-to-VEGF binding by preincubation of IVIg
with different concentrations of VEGF in the sandwich ELISA

| VEGF | Absorbance | |
|---|---|---|
| concentration | 450 nm | % inhibition |
| 0 | 1.249 | |
| 0.1 | 1.044 | 16.41 |
| 0.5 | 1.001 | 19.86 |
| 1 | 0.835 | 33.15 |
| 2 | 0.141 | 88.71 |
| 4 | 0.105 | 91.59 |
| 8 | 0.059 | 95.28 |

Concentration of IVIg was 12.5 mg/ml EC₅₀ value )

### Example 4

### Blocking of Binding to a Specific VEGF Epitope

To estimate binding potential of IVIG to the specific epitope on VEGF, either inhibition of IVIG-to-VEGF binding by anti-VEGF monoclonal antibody or inversely, inhibition of anti-VEGF monoclonal antibody to VEGF binding by IVIG, were carried out. For this purpose, in one set of experiments immobilized VEGF was saturated with anti-VEGF monoclonal antibody (inhibitor), before adding the biotinylated IVIG, while in the other one, IVIG was used as inhibitor and subjected to the solid face attached VEGF before adding the biotinylated anti-VEGF monoclonal antibody. The percent of inhibitions were calculated and presented in FIG. 4. As shown, inhibition rates of anti-VEGF monoclonal antibody-to-VEGF binding were 17.81, 10.02, 3.90 and 2.41%, when IVIG was used as an inhibitor at concentrations of 5, 2, 1 and 0.1 mg/ml, respectively (FIG. 4). On the other hand, anti-VEGF monoclonal antibody at concentrations of 20, 10 and 1 μg/ml demonstrated much higher, but still partially inhibitory activities (37.20, 28.46, and 23.37%, respectively) were found. The same set of blocking of binding-to-VEGF experiments was performed also by Western blot analysis. Results obtained confirmed that there are specific bindings of both IVIG and anti-VEGF monoclonal antibody to the VEGF, and that each of them may partially blocked binding potential of another one.

### Example 5

### Competition for Binding to VEGF in an Immunoblot

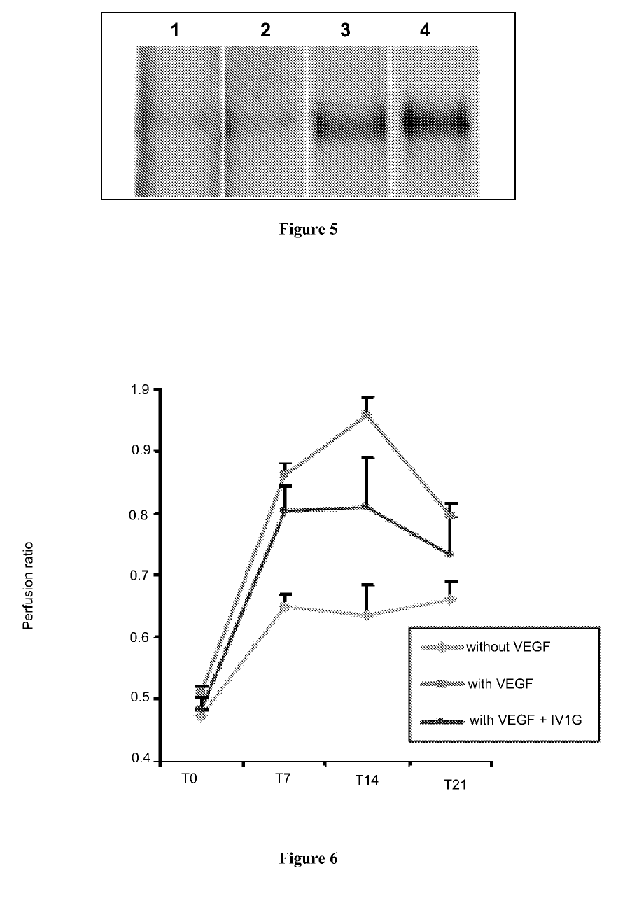

To confirm the competition of binding between a monoclonal anti-VEGF antibodies and IVIG for VEGF, the competition was performed by immunoblot (FIG. 5). After electrophoresis and blotting of the VEGF as described above (as used in Example 1 and the immunoblot in FIG. 2B), a competition experiment was performed. In Lane 1, the membrane was pre-incubated with a commercial monoclonal anti-VEGF antibody (10 μg/ml) followed by IVIg (2 mg/ml). In Lane 2, the membrane was pre-incubated with IVIG (2 mg/ml) followed by commercial monoclonal anti-VEGF antibody (10 μg/ml). Lane 3 shows direct binding of IVIG (2 mg/ml) to the membrane, and Lane 4 direct binding of commercial monoclonal anti-VEGF antibody (10 μg/ml). In Lanes 1 and 2, pre-incubation with one of either the commercial monoclonal anti-VEGF antibody or IVIG blocked binding by the other, demonstrating the presence of VEGF-binding antibodies in IVIG.

### Example 6

### Anti-VEGF Activity of IVIG in a Model of Hind-Limb Ischemia

To demonstrate the anti-angiogenic activity of IVIG in vivo, either through binding to VEGF or to VEGF receptors, the effect of IVIG administration in a peripheral ischemia model was investigated. Peripheral ischemia was induced in one hindlimb of normal mice via excision of the femoral artery. An incision was made in the middle portion of the hindlimb and the femoral artery dissected out up to the saphenous artery. The proximal and distal segments were ligated and the artery and all of its side branches excised. Animals were administered VEGF or both VEGF and IVIG, as compared to untreated animals. Mice were anesthetized and scanned with the Laser Doppler Imaging system on days 0, 7, 14 and 21 post-surgery to quantitate angiogenesis. Results were expressed as the ratio between the perfusion of the ischemic hindlimb and the non-ischemic hindlimb, with a value of 1 representing normal flow. As shown in FIG. 5, on days 7, 14 and 21 mice treated with VEGF had a higher perfusion rate than untreated animals, and the co-administration of IVIG with VEGF reduced the perfusion ratio at all time points. Thus, IVIG is capable of inhibiting angiogenesis in vivo.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method for inhibiting angiogenesis in a subject, comprising the step of administering to the subject a preparation of IVIG or fragments thereof in an amount sufficient to inhibit metastasis, whereby said preparation binds or inhibits VEGF.

2. A method for inhibiting vascular proliferation and vascular permeability in a subject, comprising the step of administering to the subject a preparation of IVIG or fragments thereof in an amount sufficient to inhibit metastases whereby said preparation binds or inhibits VEGF.

3. The method of claim 1 or 2, whereby the fragment is Fc, Fab, F(ab′), F(ab′)₂, scFv or a combination thereof.

4. The method of claim 1 or 2, whereby the preparation or fragments thereof is administered intravenously, intracavitarily, subcutaneously, intratumorally, or a combination thereof.

5. The method of claim 1 or 2, further comprising subjecting the subject to at least one other treatment modality, prior to, during or after the administration of the preparation of IVIG or fragments thereof.

6. The method of claim 5, whereby the other treatment modality is chemotherapy, immunotherapy, radiation therapy, surgery or a combination thereof.

7. A composition for inhibiting neovascularization comprising a preparation of IVIG or fragments thereof, and a pharmaceutically acceptable carrier, excipient, flow agent, processing aid, diluent or a combination thereof, wherein said preparation or fragments thereof is specific against VEGF.

8. The composition of claim 7, wherein said composition is in a form suitable for oral, intravenous, intratumoral, intraarterial, intramuscular, subcutaneous, parenteral, transmucosal, transdermal, ocular or topical administration.

9. The composition of claim 7, further comprising an additional anti-VEGF antibody.

10. The composition of claim 9, wherein the additional anti-VEGF antibody is bevacizumab.

11. The composition of claim 9, wherein the additional anti-VEGF antibody is a humanized-rat monoclonal antibody specific against VEGF (ranibizumab).

12. A method of inhibiting a tumor growth in a subject, comprising the step of administering to the subject the composition of claim 7.

13. A method of treating neovascular disorder in a subject, comprising the step of administering to the subject the composition of claim 7.

14. The composition of claim 7, wherein the preparation of IVIG or fragments thereof is isolated from the plasma of a pool of subjects, wherein said pool of subjects are in cancer remission, healthy or a combination thereof.

15. The method of claim 13, whereby treating comprises inhibiting, suppressing, delaying, alleviating symptoms, reducing symptoms, reducing incidence of, or a combination thereof.

16. The method of claim 13, whereby treating is curing.

17. A method for inhibiting angiogenesis in a subject, comprising the step of administering to the subject a preparation of IVIG or fragments thereof in an amount sufficient to inhibit metastasis, whereby said preparation or fragments thereof inhibits VEGF receptor.

18. A method for inhibiting vascular proliferation, vascular permeability or their combination in a subject, comprising the step of administering to the subject a preparation of IVIG or fragments thereof in an amount sufficient to inhibit metastases whereby said preparation or fragments thereof inhibits VEGF receptor.

19. The method of claim 17 or 18, whereby the VEGF receptor is VEGFR-1, VEGFR-2, VEGFR-3 or a combination thereof

20. The method of claim 17 or 18, whereby the fragment is Fc, Fab, F(ab′), F(ab′)₂, scFv or a combination thereof.

21. The method of claim 17 or 18, whereby the preparation or fragments thereof is administered intravenously, intracavitarily, subcutaneously, intratumorally, or a combination thereof.

22. The method of claim 17 or 18, further comprising subjecting the subject to at least one other treatment modality, prior to, during or after the administration of the preparation of IVIG or fragments thereof.

23. The method of claim 22, whereby the other treatment modality is chemotherapy, immunotherapy, radiation therapy, surgery or a combination thereof.

24. A composition for inhibiting neovascularization comprising a preparation of IVIG or fragments thereof, and a pharmaceutically acceptable carrier, excipient, flow agent, processing aid, diluent or a combination thereof, wherein said preparation or fragments thereof is specific against a VEGF receptor.

25. The composition of claim 24, wherein said composition is in a form suitable for oral, intravenous, intratumoral, intraarterial, intramuscular, subcutaneous, parenteral, transmucosal, transdermal, ocular or topical administration.

26. The composition of claim 24, further comprising an additional anti-VEGF antibody.

27. The composition of claim 26, wherein the additional anti-VEGF antibody is bevacizumab.

28. The composition of claim 26, wherein the additional anti-VEGF antibody is a humanized-rat monoclonal antibody specific against VEGF (ranibizumab).
